(19) United States
(12) Patent Application Publication
(10) Pub. No.: US 2010/0008886 A1
(43) Pub. Date: Jan. 14, 2010

(54) APPLICATION OF THE OIL FRACTION OBTAINED FROM SOUR CHERRY (PRUNUS CESARUS) SEED KERNEL

(76) Inventors: Árpád Tósaki, Debrecen (HU); Miklós Vecsernyés, Debrecen (HU); László Fésüs, Debrecen (HU); István Bak, Debrecen (HU); Béla Juhász, Debrecen-Jozsa (HU); László Papp, Fehergyarmat (HU); Szilvia Toth, Debrecen (HU)

Correspondence Address: BROWDY AND NEIMARK, P.L.L.C.;624 NINTH STREET, NW, SUITE 300, WASHINGTON, DC, 20001-5303, US

(21) Appl. No.: 12/442,005

(22) PCT Filed: Sep. 14, 2007
(86) PCT No.: PCT/HU07/00085

§ 371 Date: Mar. 19, 2009

(30) Foreign Application Priority Data
Sep. 22, 2006 (HU) P 06 00740

(51) Int. Cl. A61K 8/92; A61K 8/97; A61Q 5/00; A61Q 19/08

(52) U.S. Cl. 424/74; 424/735

(57) ABSTRACT

The present invention relates to the use of the oil components of sour cherry (Prunus cerasus) kernel. Particularly, the application relates to the use of the oil fraction obtained from the seed of Prunus cerasus (sour cherry) as a component of cosmetics or drugs. The invention also relates to the use of the oil components for the preparation of cosmetics or drugs and to cosmetics and drugs containing the oil fraction as active ingredient alone or in combination with other active substances.

The present invention relates to the use of the oil components of sour cherry (Prunus cerasus) kernel. Particularly, the application relates to the use of the oil fraction obtained from the seed of Prunus cerasus (sour cherry) as a component of cosmetics or drugs. The invention also relates to the use of the oil components for the preparation of cosmetics or drugs and to cosmetics and drugs containing the oil fraction as active ingredient alone or in combination with other active substances.

Nowadays more and more people recognize the importance of healthy living and make efforts to maintain their organism in perfect condition as long as possible. They try to use and consume the utmost natural substances. Particularly popular among the natural substances are fruits and vegetables containing several substances essential for the organism, like for example vitamins. Such fruits and vegetables and the preparations obtained from the same are highly suitable for administering valuable active substances into the organism. This can be achieved in various ways like for example in the form of preparations for external applications, preparations for internal application or preparations suitable for human consumption.

Sour cherry (Prunus cerasus) is a very wide-spread and important fruit in the world. Although the relevant prior art contains several references in connection with the use of various substances obtained from the components of sour cherry (fruit, stalk etc.) no mention in connection with the most important part thereof, the sour cherry seed can be found.

The inventors also recognized that a certain part of the extract obtained from the sour cherry seed exerts high cardioprotective activity in certain biological samples without any side effects (International Patent Application No. PCT/HU2006/000093 based on Hungarian Patent Application No. P0500975).

The sour cherry seed extract, however, can be divided into two parts: a solid fraction and an oil fraction. Inventors found that the above mentioned cardioprotective effect is triggered mostly by the active substances of the solid phase.

Surprisingly, we found that not only the active substances of the solid phase, but those of the oil phase also possess beneficial biological properties. We also found that these substances are highly suitable for the use in cosmetics and drugs.

The prior art does not contain any document describing the isolation of the components of the sour cherry kernel, or suggesting that the components of the oil fraction of the sour cherry seed kernel could be successfully used as cosmetics or drugs.

Accordingly, the aim of the present invention is to develop new possibilities for the use of the oil fraction of the sour cherry seed kernel.

According to the above, the present invention relates to novel uses of the sour cherry seed. More precisely, the invention relates to the use of the oil fraction of the sour cherry seed in cosmetics or drugs. The invention also relates to the use of the oil components for the preparation of cosmetics or drugs and to cosmetics and drugs containing the oil fraction as active ingredient alone or in combination with other active substances.

The separation procedure, after removing the wall of sour cherry (Prunus cerasus) stone, leads to Fraction I (oil fraction) and Fraction II (solid phase). Separation of the oil fraction is achieved by the use of n-hexane (see below). We wish to mention that obtaining the oil phase is also possible by cold pressing process.

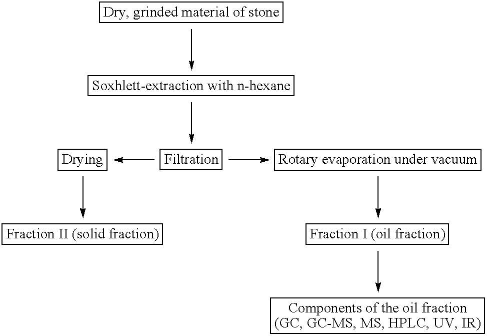

The sour cherry seed contains two main fractions:

Fraction I: The sour cherry seed kernel contains about 30-33% of oil fraction (“O”) including vitamin E (alpha-tocopherol, 52 mg/100 g), vitamin E-like components (delta-tocopherol, tocotrienol), unsaturated free fatty acid esters (hexa-, hepta-, and octadecane acids, aldehide (e.g., hexanal), mixtures of triglycerids including free fatty acids LLL (L: linoleic acid) LLO (oleic acid), LLP (P: palmitil acid). The total tocopherol content of the “O” fraction of sour cherry seed kernel is about 90 mg/100 g. The “O” fraction does not contain flavonoids, polyphenols, and cyanide components in comparison with the Fraction II (solid fraction). However, the “O” fraction has strong antioxidant activity (based on the components of the kernel oil).

The “O” fraction can be obtained by n-hexane extraction from the sour cherry seed kernel. However, for the production of “body care”, “face lifting” creams, and other beauty products (e.g., soap, deodorant) for commercial distribution, the “O” fraction will be directly got by cold press from the sour cherry seed kernel (after removing the wall). Thus, we can avoid even the minimal n-hexane contamination, as an organic agent, in the final commercial products.

Fraction II: the solid (S) fraction of sour cherry seed includes flavonoids, rhamnetin, malvidin, delfinidin, pinocembrin, naringenin, quercetin, rezveratrol, dihydroquercetin, peonidin, apigenin, pro- and athocyanidins, glucose (e.g., feruloil-D-glucose, cumaroil-glucose, feruloil-d-glucose), stilbenes, catechins, gallic acid, gallocatechins, and other atioxidants (e.g., gallotannin). The analysis and application of Fraction II (solid fraction) are the subject of another patent applications (International Patent Application No. PCT/HU2006/000093 based on Hungarian Patent Application No. P0500975).

### Uses of the Fractions

The oil fraction of the sour cherry seed kernel, which contains vitamin E, vitamin E-like components (tocotrienols), and unsaturated fatty acids can be initially used as a non prescribed product against dehydration, wrinkle reduction, aging, UV irradiation, or as a cream of beauty (e.g., body care, soap, face lifting creams), and later a prescribed product can be developed against eczema and various dermatitis. It's also possible that an orally used capsule of the oil fraction can be developed for therapy. However, the antioxidant properties of the “O” fraction are also can improve blood circulation in the skin and other vital organs.

The solid fraction of the sour cherry seed (stone) kernel contains various flavonoids and citoprotective natural molecular structures. The composition of the sour cherry seed kernel is effective against the ischemia/reperfusion-induced myocardial damage. The protective effect of sour cherry seed (solid fraction, or oil-free fraction) is about 50% less in comparison with those of beta-blockers or calcium channel antagonists used in cardiovascular therapy. However, the reverse (side) effects of the sour cherry seed kernel (oil-free fraction) cannot be detected between 5 mg/kg and 500 mg/kg doses in rodents (rats and mice). Thus, the combination of sour cherry seed kernel with a calcium channel blocker or beta receptor blocker can be effective by reducing their (calcium channel or beta blockers) reverse effects in the cardiovascular therapy. The invention of the solid fraction (S, or oil-free fraction) has been described and protected by previous patent applications (International Patent Application No. PCT/HU2006/000093 based on Hungarian Patent Application No. P0500975).

Cosmetics and drugs containing the oil fraction obtained from sour cherry seed kernel can be prepared by using methods well known a person skilled in the art. The cosmetics and drugs thus obtained may contain other active substances in addition to the oil fraction and the usual additives resulting optionally in a synergistic effect. Such active ingredients for example antiseptics or hydrating agents are well known for a person skilled in the art.

A great advantage of the solution of the present invention is that the active ingredients of the oil fraction do not contain any component harmful to the organism and no side effects during the use occur.

### RESULTS: ANALYSIS (COMPONENTS) OF THE OIL FRACTION OF SOUR CHERRY (PRUNUS CERASUS) SEED

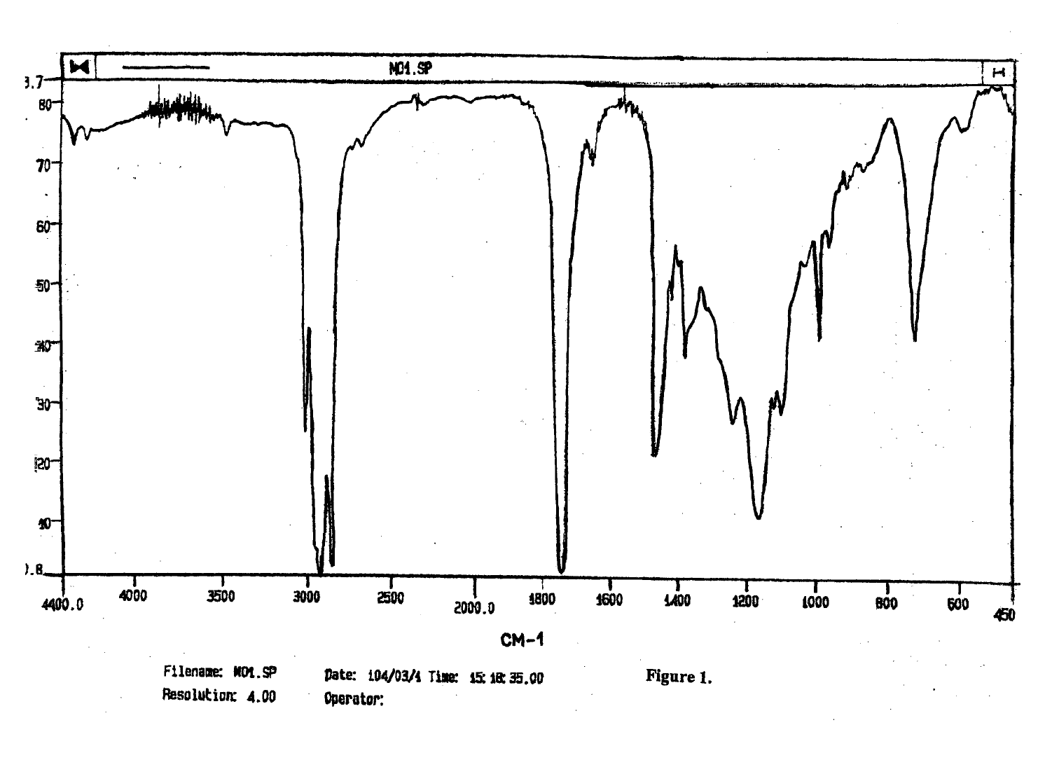

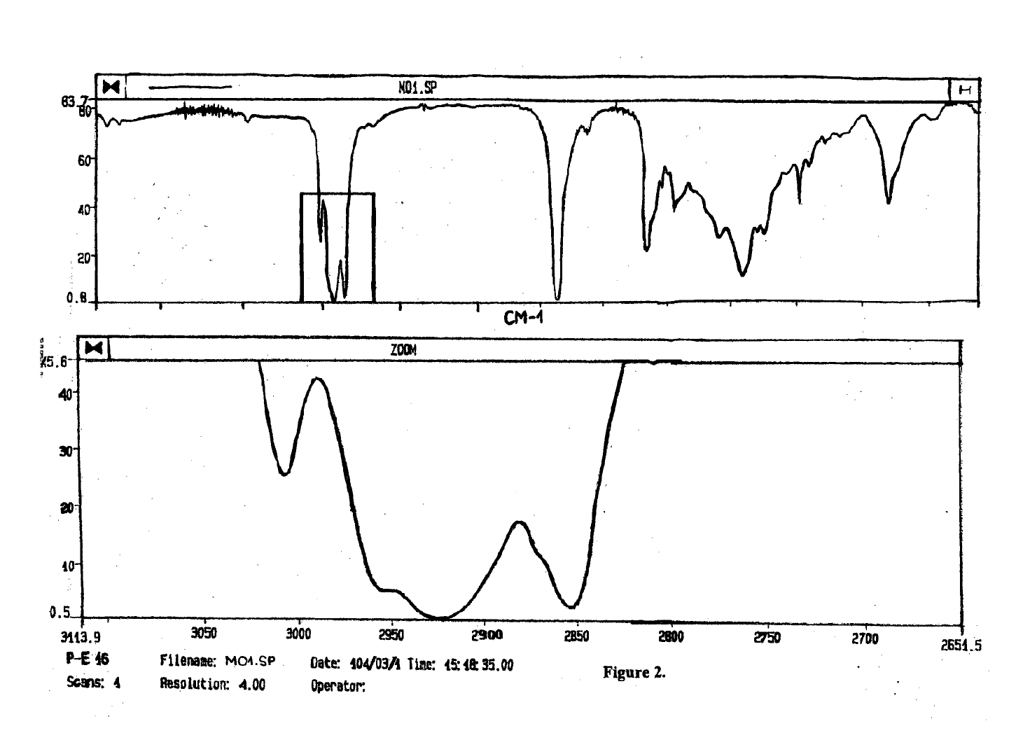

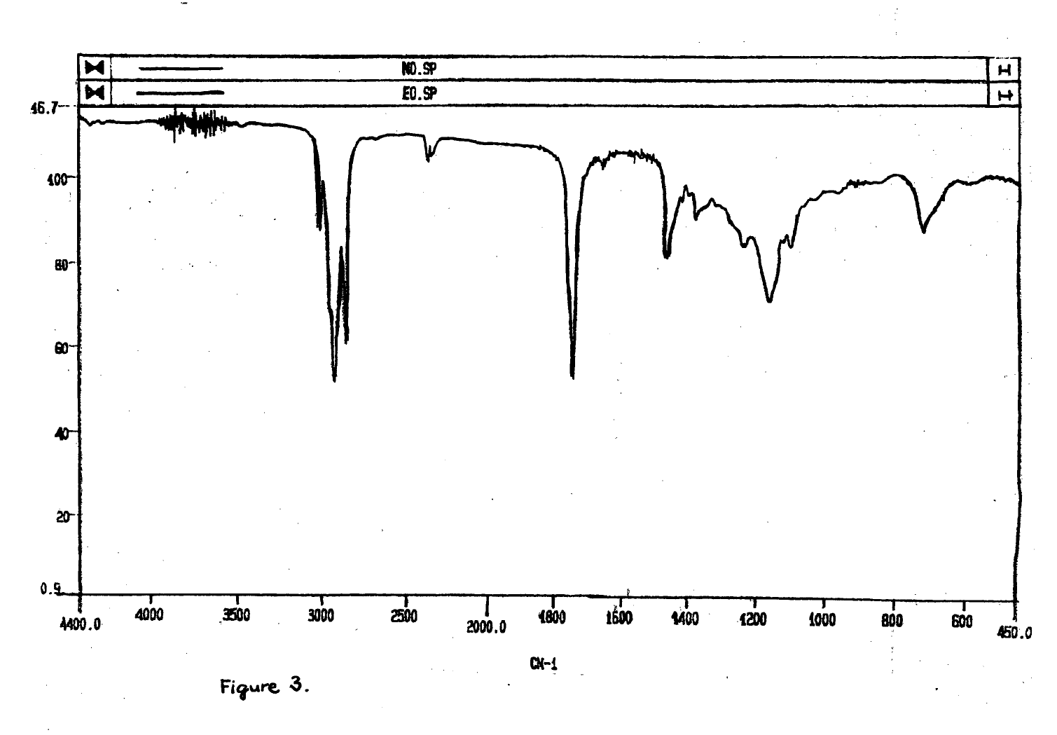

FIG. 1, FIG. 2, and FIG. 3 show the infra red (IR) spectra of “O” fraction. FIG. 1 shows a typical unsaturated fatty acid ester component at 3020 cm⁻¹. An ester group (═O) can be detected at 1742 cm⁻¹ of the spectra. Between 2500 and 2800 cm⁻¹, OH⁻ group peaks are detected indicating the components of free carbonyl acids. The long carbonyl chain components can bee seen at the ranges between 1460 and 720 cm⁻¹, and 3000 and 2800 cm⁻¹

The spectra of “O” of sour cherry kernel were compared to the sunflower's oil (FIG. 3) and many similarities were found. However, the major difference between the “O” fraction of the sour cherry kernel and sunflower's oil is in the content of free fatty acids. Thus, free fatty acids can not be found (or in a very little amount) in the sunflower's oil. However, the “O” fraction of the sour cherry seed kernel contains a relatively high amount of free fatty acids detected between 970 and 930 cm⁻¹.

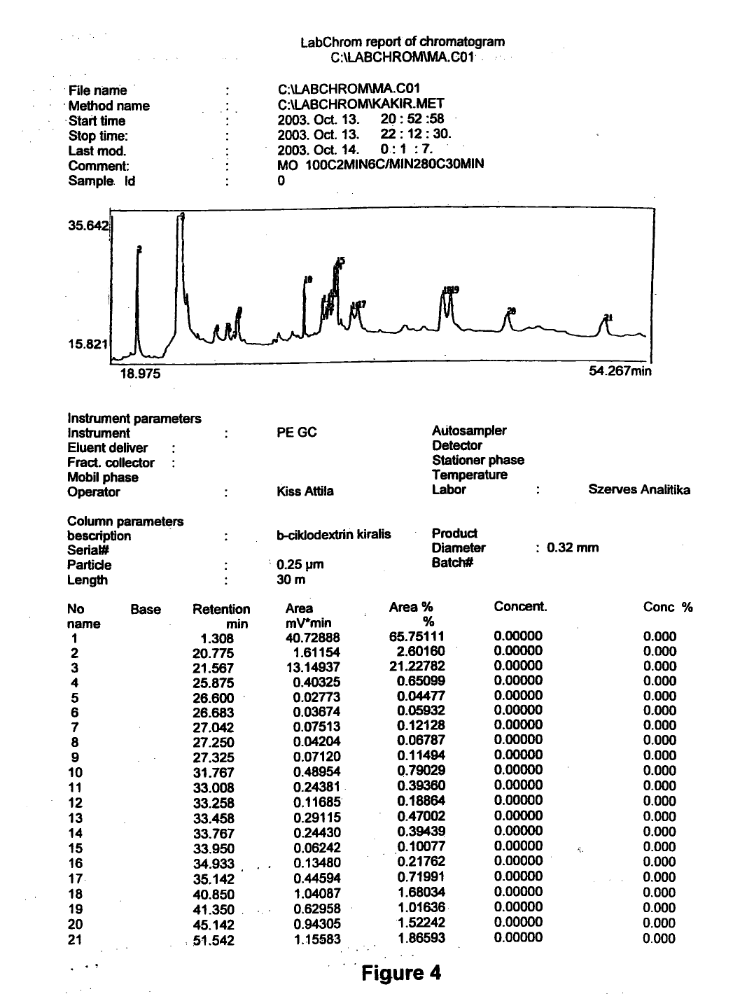

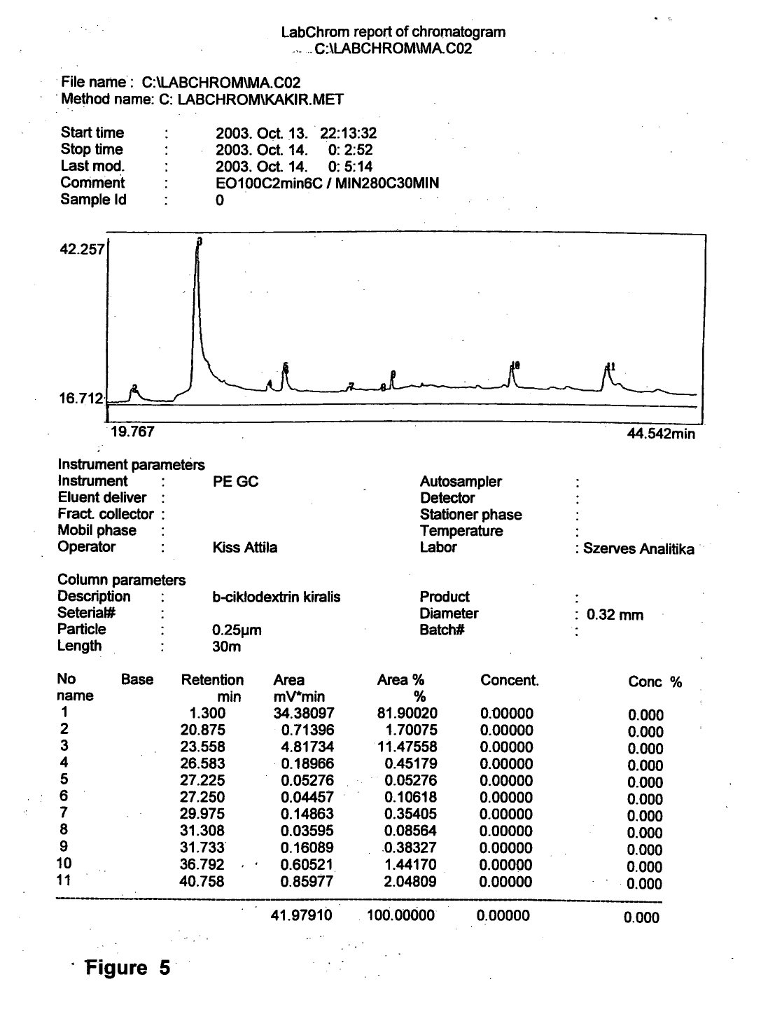

FIG. 4 shows the gas chromatogram (GC) of “O” fraction in comparison with the sunflower (FIG. 5) chromatogram. The “O” fraction of sour cherry seed kernel, beside the main components, contains many minor components (FIG. 4) in comparison with the analysis of sunflower oil (FIG. 5).

The free radical scavenger activity of “O” fraction was studied by galvinoxyl radical method. The results show that the “O” fraction possesses free radical scavenger activities.

### GC-MS Studies:

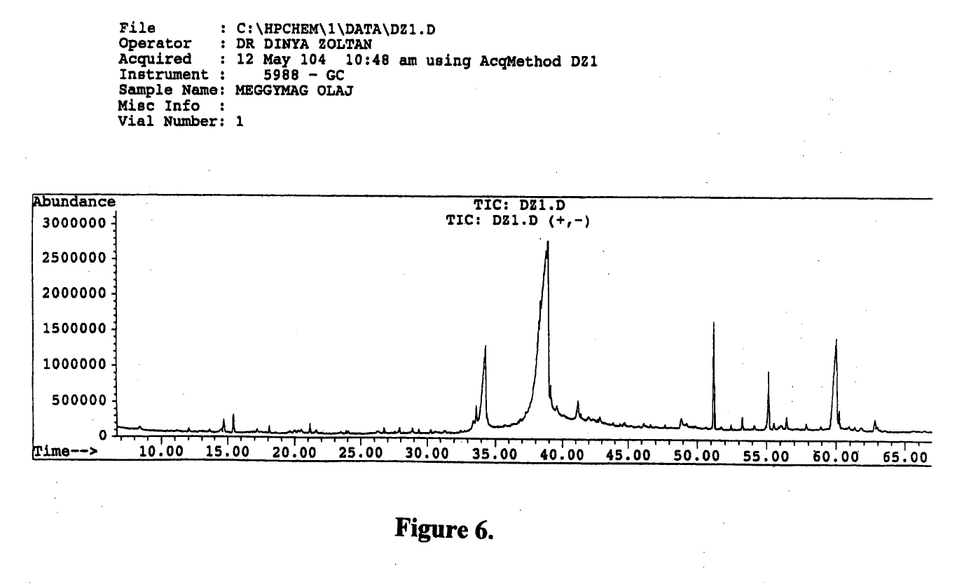

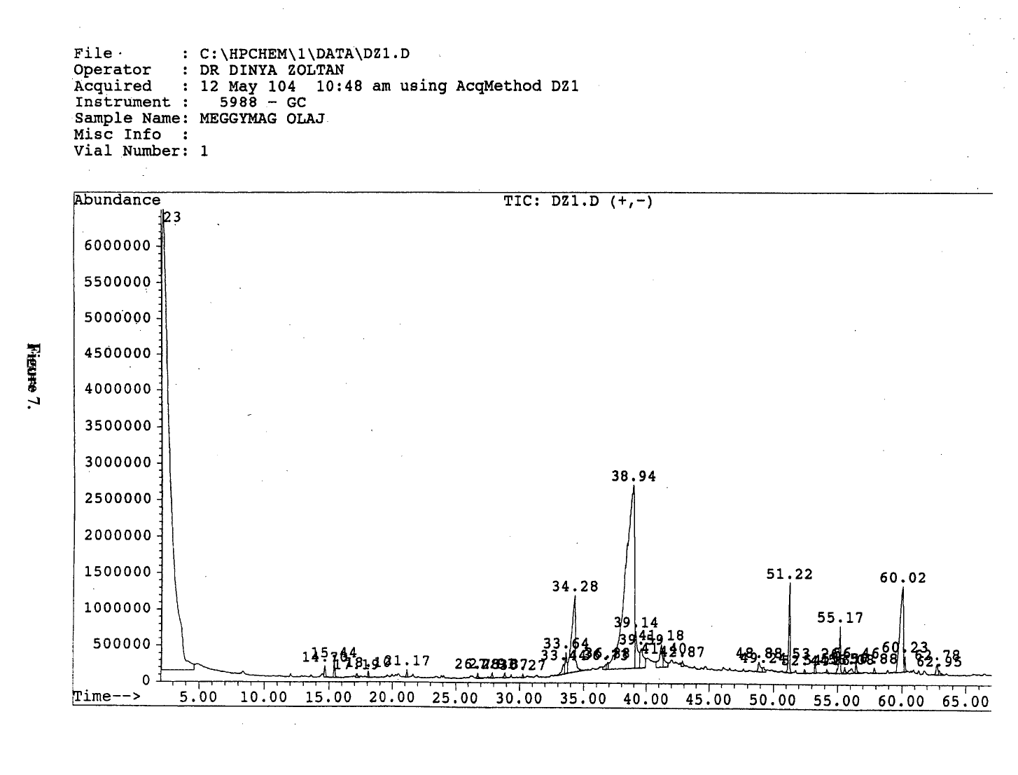

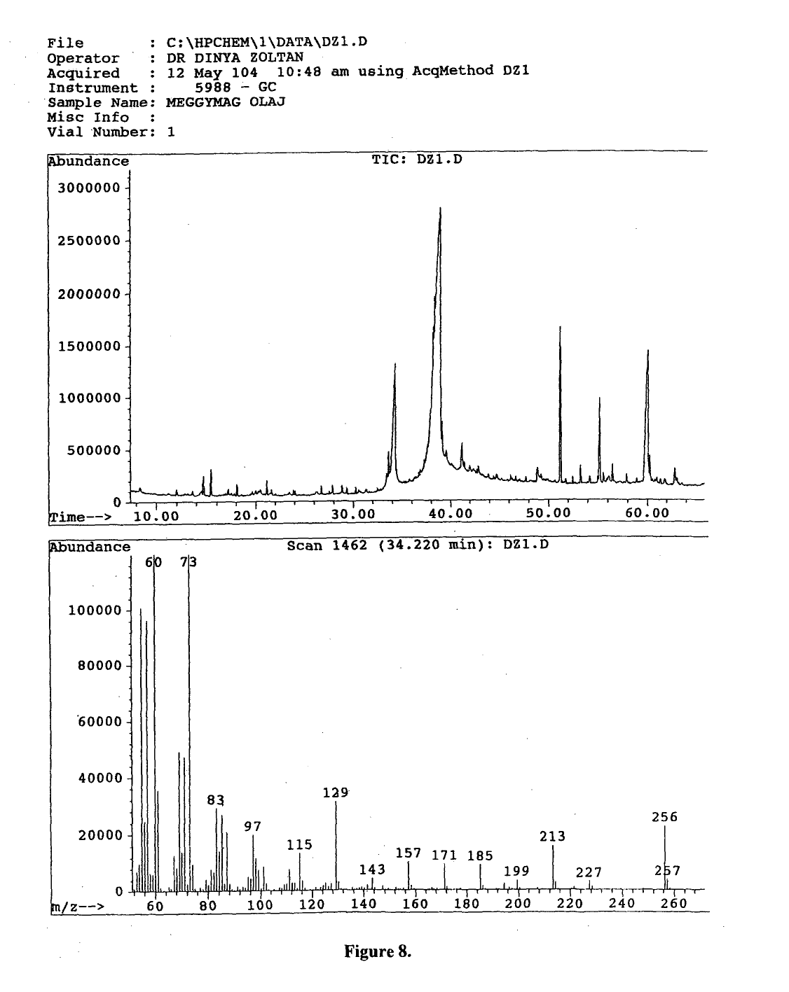

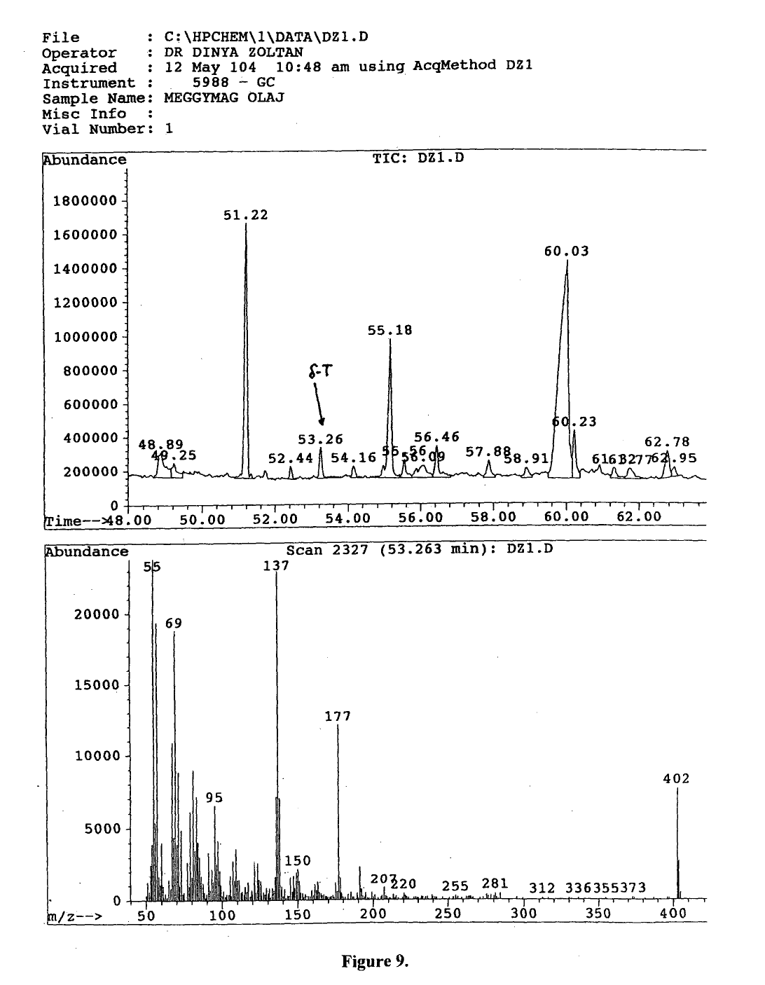

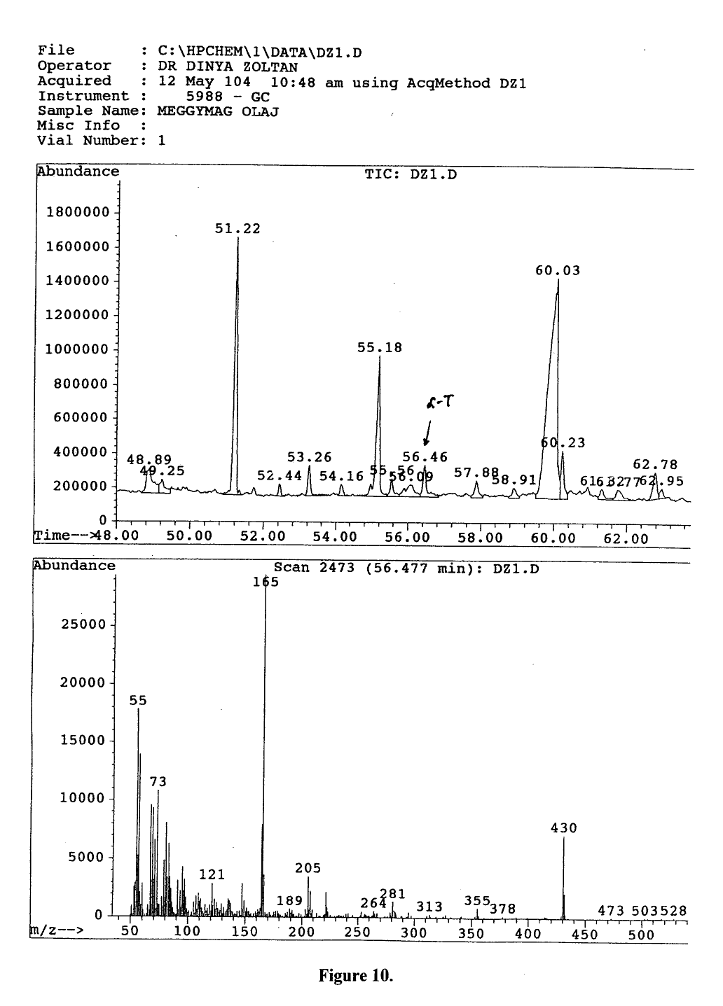

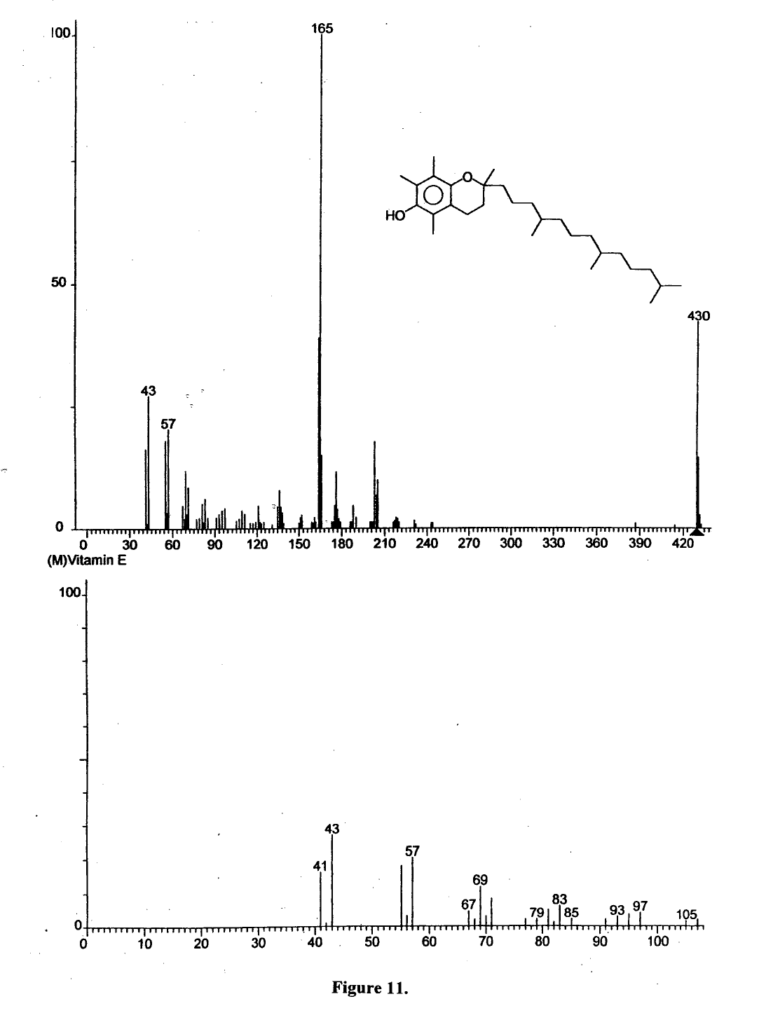

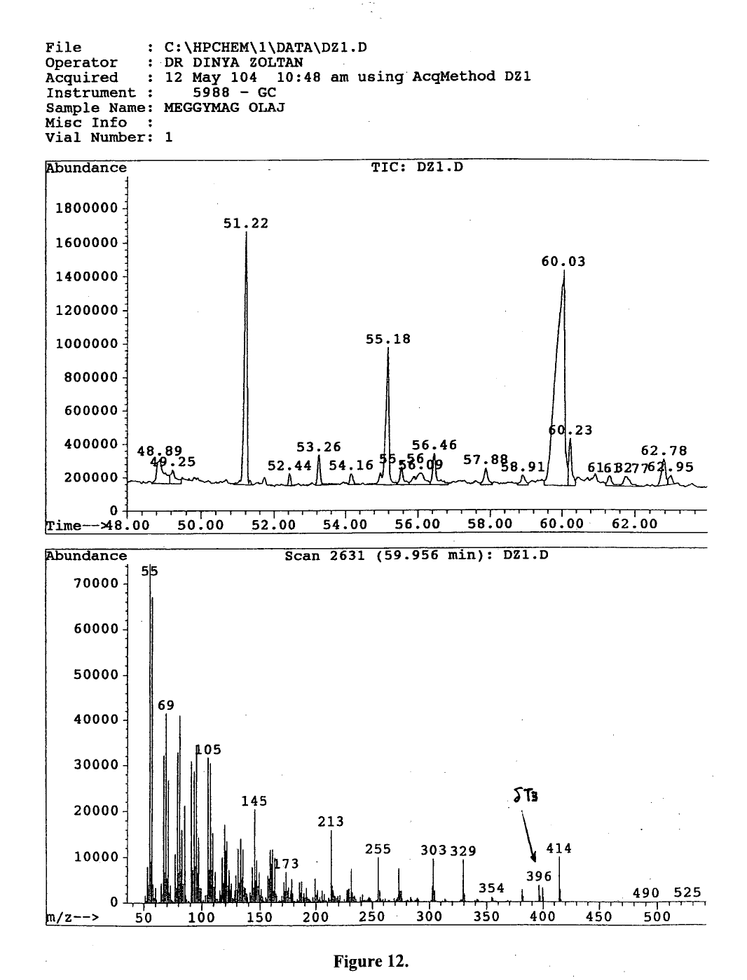

Chromatograms were obtained by total ion chromatography. FIGS. 6 and 7 show that “O” fraction consists of mainly triglycerides including linoleic acid (LA), oleic acid (OL). However, a small amount of palmitil acid and stearin acid was also detected. Thus, the “O” fraction contains mainly unsaturated triglyceride components. Beside triglycerides, free fatty acids such as ω-3 α-linoleic acid, hexa-, hepta-, octa-decanoic acids, and aldehydes (e.g., hexanal and decadienal) can also be detected in the “O” fraction (FIG. 8). The most important components of the “O” fraction are vitamin E and its isomers (FIGS. 9-12). Thus, δ-tocopherol (FIG. 9), α-tocopherol (FIGS. 10 and 11), and δ-tocotrienol (FIG. 12) are the major components. The α-tocopherol content is 52-53 mg/100 g, while the total tocopherol content is about 80-85 mg/100 g.

The oil (O) fraction can be used for ointment and soap production in order to improve blood circulation in the vasculature of skins, and prevention of arteriosclerosis in vital organs. The following components of sour cherry seed kernel (‘O’ fraction) are patented: unsaturated triglyceride components, free fatty acids such as (ω-3 α-linoleic acid, hexa-, hepta-, octadecanoic acids, and aldehydes (e.g., hexanal and decadienal), and vitamin E and its isomers (δ-tocopherol, α-tocopherol, and δ-tocotrienol) in the “O” fraction. The a-tocopherol content is 52-53 mg/100 g, while the total tocopherol content is about 80-85 mg/100 g. It is also possible (at the moment no evidence) that some stable prostaglandin derivatives are also responsible for the protective effects of the ‘O’ phase. For the production of commercial nonprescribed products, and prescribed (later) products in cardiovascular therapy, the “O” fraction will be obtained by cold press procedure in order to avoid the n-hexane contamination in samples.

1. In a method of making cosmetics, using an oil fraction the improvement wherein said oil fraction is sour cherry seed extract.

2. The method according to claim 1, wherein the cosmetic is a hydrating preparation.

3. The method according to claim 1, wherein the cosmetic is an anti-aging preparation.

4. The method according to claim 1, wherein the cosmetic is -wrinkle-reducing preparation.

5. The method according to claim 1, wherein the cosmetic is an UV-protecting preparation.

6. The method according to claim 2, wherein the preparation is an ointment.

7. The method according to claim 1, wherein the cosmetic is a shampoo.

8. A method of preparing cosmetics comprising using an oil fraction of sour cherry seeds, the cosmetics selected from the group comprising a hydrating preparation, an anti-aging preparation, a wrinkle-reducing preparation, or a shampoo.

9. Cosmetic containing the oil fraction of the sour cherry seed as active ingredient alone or in combination with other active ingredients.

10. The method according to claim 8, wherein the preparation is an ointment.

11. The method according to claim 3, wherein the preparation is an ointment.

12. The method according to claim 4, wherein the preparation is an ointment.

13. The method according to claim 5, wherein the preparation is an ointment.
